(19) United States
(12) Patent Application Publication
(10) Pub. No.: US 2024/0335820 A1
(43) Pub. Date: Oct. 10, 2024

(54) PHOTOCATALYST COMPOSITION, IODINE SOLUTION, PHOTOCATALYST DISPERSION LIQUID, ANTIBACTERIAL ACTIVATION METHOD, AND ANTIBACTERIAL ACTIVATION APPARATUS

(71) Applicant: Sharp Kabushiki Kaisha, Sakai City (JP)

(72) Inventors: Ryota TANAKA, Sakai City (JP); Shinya OKAZAKI, Sakai City (JP)

(21) Appl. No.: 18/624,780

(22) Filed: Apr. 2, 2024

(30) Foreign Application Priority Data
Apr. 7, 2023 (JP) 2023-062949

(51) Int. Cl. B01J 23/30; A61L 2/08; B01J 23/652; B01J 35/27; B01J 35/39

(52) U.S. Cl. CPC B01J 23/30; A61L 2/088; B01J 23/6527; B01J 35/27; B01J 35/39

(57) ABSTRACT

The present disclosure provides a photocatalyst composition that has excellent antibacterial properties in a bright place and can suppress a decrease in antibacterial action over time in a dark place. The present disclosure provides a photocatalyst composition containing a photocatalyst particle, a co-catalyst, and iodine or a water-soluble iodine compound, characterized in that the iodine is formed from an iodide ion by photocatalyst activity generated by the photocatalyst particle receiving light.

### BACKGROUND OF THE INVENTION

### Field of the Invention

The present disclosure relates generally to a photocatalyst composition, a photocatalyst dispersion liquid, an iodine solution, an antibacterial activation method, and an antibacterial activation apparatus.

### Description of the Background Art

A method for generating active agent is known in which titanium dioxide (photocatalyst) containing oxoacid is irradiated with ultraviolet light to release active agent. The release of such active agent results in antibacterial action.

### Technical Problem

However, in conventional photocatalyst compositions, photocatalyst activity produces antibacterial action in the light, but photocatalyst activity does not occur in the dark. If the photocatalyst composition contains antibacterial agent, the photocatalyst composition can have antibacterial action in the dark, but over time the amount of antibacterial agent decreases and the antibacterial action also decreases.

### SUMMARY OF THE INVENTION

The present disclosure has been made in view of these circumstances, and provides a photocatalyst composition that has excellent antibacterial properties in the bright place and can suppress the decrease in antibacterial action over time in the dark.

### Solution to Problem

The present disclosure provides a photocatalyst composition containing a photocatalyst particle, a co-catalyst, and iodine or a water-soluble iodine compound, characterized in that iodine is formed from an iodide ion by photocatalyst activity generated by the photocatalyst particle receiving light.

### Advantageous Effects of Invention

In the photocatalyst composition of the present disclosure, iodine (12) is formed by the photocatalyst activity generated by the photocatalyst particle receiving light. Although details are not known, the present inventors believe that iodine reacts with water to form highly reactive H₂OI⁺. This H₂OI⁺ takes an electron away from a bacterium it contacts to deactivate (oxidize) the bacterium. Thus, the photocatalyst composition of the present disclosure has excellent antibacterial properties even in the dark. In addition, the photocatalyst composition of the present disclosure is considered to have, in the light, excellent antibacterial properties due to both antibacterial actions by photocatalyst activity and by H₂OI⁺.

When H₂OI⁺ takes an electron away from a bacterium, etc., an iodide ion (I⁻) is formed. This iodide ion is oxidized to iodine by photocatalyst activity in the light, so that even if iodine is consumed by the antibacterial action in the dark, the amount of iodine can be regenerated in the light. Therefore, the photocatalyst composition of the present disclosure can prevent the antibacterial action in the dark from decreasing over time. Not limited to regeneration of the antibacterial action, the photocatalyst composition is also effective in regenerating antiviral, antifungal, and anti-allergen actions.

### BRIEF DESCRIPTION OF THE DRAWINGS

FIG. 1 is a schematic diagram of a photocatalyst composite contained in a photocatalyst composition according to an embodiment of the present disclosure.

FIG. 2A is a schematic cross-sectional view of a photocatalyst dispersion liquid.

FIG. 2B is a schematic cross-sectional view of a substrate covered with a photocatalyst coating layer.

FIG. 2C is a schematic cross-sectional view of a photocatalyst film.

FIG. 3 is an illustration of an antibacterial action of the photocatalyst composite in the dark and regeneration of iodine in the light.

FIG. 4 is an illustration of a reaction in which iodine is formed from an iodate ion by photocatalyst activity.

FIG. 5 is a schematic cross-sectional view of an antibacterial activation apparatus according to an embodiment of the present disclosure.

FIG. 6 is Table 1 showing compositions of photocatalyst dispersion liquids and evaluations.

FIG. 7 is Table 2 showing compositions of photocatalyst dispersion liquids and evaluations.

### DETAILED DESCRIPTION

The photocatalyst composition according to the present disclosure contains a photocatalyst particle, a co-catalyst, and iodine or a water-soluble iodine compound, characterized in that iodine is formed from an iodide ion by photocatalyst activity generated by the photocatalyst particle receiving light.

The water-soluble iodine compound is preferably metal iodide, ammonium iodide, iodic acid, or metal iodate. A hole generated by the photocatalyst particle receiving light can oxidize an iodide ion to form iodine, which can regenerate antibacterial agent.

The co-catalyst is preferably supported on the surface of the photocatalyst particle. An electron generated by the photocatalyst particle receiving light can be consumed by the co-catalyst, and recombination of the electron and the hole can be suppressed.

The iodine or water-soluble iodine compound is preferably supported on the surface of the photocatalyst particle.

The photocatalyst particle preferably contains tungsten oxide. The tungsten oxide generates a hole with high oxidizing power by receiving visible light, which can increase the regeneration effect of the antibacterial agent.

The co-catalyst is preferably a metal or metal oxide containing at least platinum. An electron generated by the photocatalyst particle receiving light can be consumed by the co-catalyst, and recombination of the electron and the hole can be suppressed.

A ratio (B/A) of weight B of the co-catalyst to weight A of the photocatalyst particle in the photocatalyst composition is preferably (0.01/100) or more and (1/100) or less.

A ratio (D/C) of weight D of the iodine or water-soluble iodine compound to weight C of the photocatalyst particle in the photocatalyst composition is preferably (0.1/100) or more and (5/100) or less. The present disclosure also provides an iodine solution containing iodine, which is an extract of the photocatalyst composition of the present disclosure after light irradiation.

The present disclosure also provides photocatalyst dispersion liquid containing a photocatalyst particle, a co-catalyst, iodine or a water-soluble iodine compound, and a dispersion medium. By applying and drying the photocatalyst dispersion liquid of the present disclosure on a substrate, a photocatalyst coating layer can be formed, and gas decomposition action, antibacterial action, etc. can be expressed on the surface of the substrate.

The percentage of the photocatalyst particle in the photocatalyst dispersion liquid is preferably 0.1 wt % or more and 30 wt % or less.

It is preferred that the photocatalyst dispersion liquid of the present disclosure further contains a pH adjuster. The pH of the photocatalyst dispersion liquid of the present disclosure is preferably 1 or more and 7 or less, and is more preferably 2 or more and 5 or less.

The present disclosure also provides an antibacterial activation method including irradiating a photocatalyst composition containing a photocatalyst particle, a co-catalyst, and a water-soluble iodine compound with light, characterized in that iodine is formed from an iodide ion by photocatalyst activity generated by the photocatalyst particle receiving light.

The antibacterial activation method of the present disclosure preferably further includes extracting iodine from the photocatalyst composition after light irradiation. For example, iodine is extracted to be used for an iodine additive for adding the extracted iodine to the water in the aquarium.

The present disclosure also provides an antibacterial activation apparatus including a light source device provided to irradiate light to a photocatalyst composition containing a photocatalyst particle, a co-catalyst, and a water-soluble iodine compound, characterized in that iodine is formed from an iodide ion by photocatalyst activity generated by the photocatalyst particle receiving light irradiated from the light source device.

An embodiment of the present disclosure will be described below with reference to the drawings. The configurations shown in the drawings and the following description are examples, and the scope of the present disclosure is not limited to those shown in the drawings and the following description.

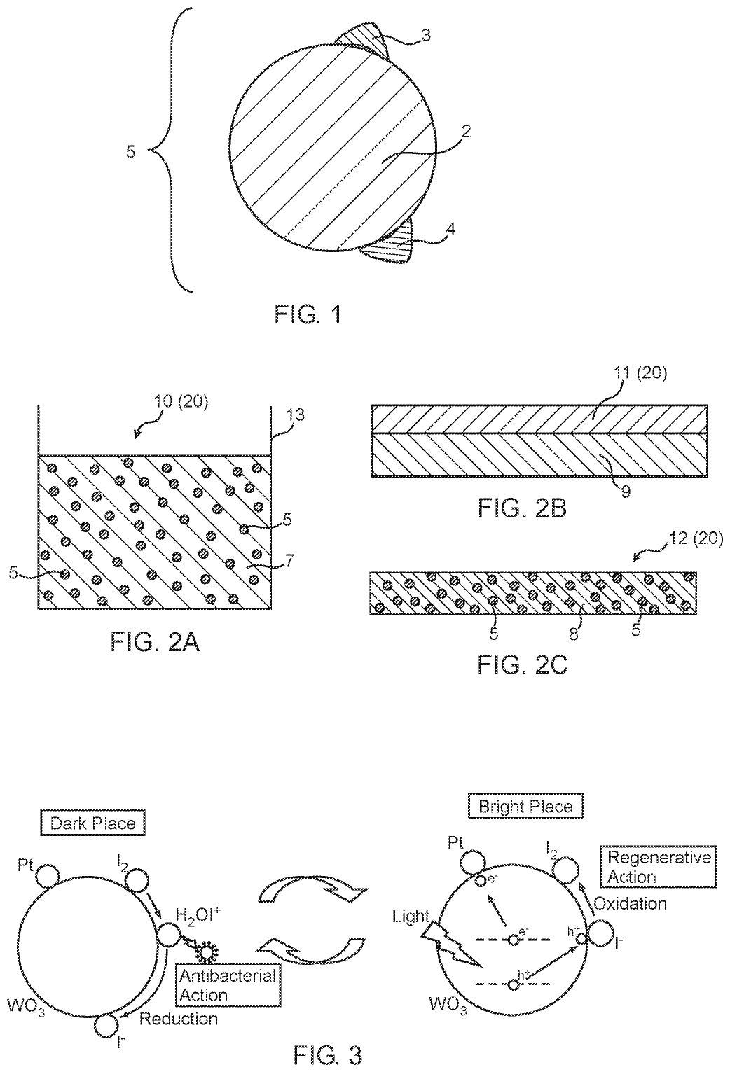

FIG. 1 is a schematic diagram of a photocatalyst composite contained in a photocatalyst composition of the present embodiment, FIG. 2A is a schematic cross-sectional view of a photocatalyst dispersion liquid 10, FIG. 2B is a schematic cross-sectional view of a substrate 9 covered with a photocatalyst coating layer 11, and FIG. 2C is a schematic cross-sectional view of a photocatalyst film 12.

The photocatalyst composition 20 of the present embodiment contains a photocatalyst particle 2, a co-catalyst 3, and iodine or a water-soluble iodine compound, and is characterized in that iodine is formed from an iodide ion by photocatalyst activity generated by the photocatalyst particle 2 receiving light.

The photocatalyst composition 20 is a composition containing a photocatalyst, such as the photocatalyst dispersion liquid 10 as shown in FIG. 2A, the photocatalyst coating layer 11 as shown in FIG. 2B, and the photocatalyst film 12 as shown in FIG. 2C. The photocatalyst composition 20 may be a powder containing a photocatalyst composite 5.

The photocatalyst composition 20 can include the photocatalyst composite 5 as shown in FIG. 1. The photocatalyst composite 5 can have a configuration in which the co-catalyst 3 and iodine (I₂) or a water-soluble iodine compound 4 are supported on the surface of the photocatalyst particle 2. If the photocatalyst composition 20 is the photocatalyst dispersion liquid 10 and a dispersion medium 7 is an aqueous liquid, the iodine or water-soluble iodine compound 4 may be dissolved in the dispersion medium 7 without being supported on the surface of the photocatalyst particle 2.

The photocatalyst particle 2 is not particularly limited as long as it is a particle that exhibits photocatalyst activity when receiving light, such as a tungsten oxide particle or a titanium oxide particle (TiO₂), preferably being a tungsten oxide particle with visible light responsiveness. The tungsten oxide particle, as long as having photocatalyst activity, may be a tungsten oxide particle with a composition deviating from a stoichiometric composition. The tungsten oxide particle may also contain an impurity atom or additive atom to the extent that photocatalyst activity is not lost.

Examples of tungsten oxide contained in the tungsten oxide particle include WO₃(tungsten trioxide), WO₂, WO, W₂O₃, W₄O₅, W₄O₁₁, W₂₅O₇₃, W₂₀O₅₈, and W₂₄O₆₈, and mixtures thereof. WO₃ is preferred for tungsten oxide to improve photocatalyst activity.

A crystal structure of tungsten oxide contained in the tungsten oxide particle is not particularly limited. Examples of the crystal structure of tungsten oxide include monoclinic, triclinic, orthorhombic, and mixed crystals of at least two thereof.

The photocatalyst composition 20 may contain only one type of tungsten oxide particle or two or more types of tungsten oxide particles as the photocatalyst particle 2. A photocatalyst contained in the photocatalyst composition 20 can be mainly composed of a tungsten oxide particle. However, the photocatalyst composition 20 may further contain a photocatalyst particle other than the tungsten oxide particle in addition to the tungsten oxide particle, as the photocatalyst particle 2.

An average particle diameter D50 of the photocatalyst particle 2 (primary particle) is preferably 5 nm or more and 500 nm or less, and is more preferably 5 nm or more and 200 nm or less. The particle diameter can be measured by a BET specific surface area meter, laser diffraction type particle size distribution meter, dynamic light scattering type particle size distribution meter or the like.

The co-catalyst 3 is a catalyst that promotes a chemical reaction caused by the photocatalyst particle 2 receiving light. The co-catalyst 3 may be supported on the photocatalyst particle 2. By loading the co-catalyst 3 onto the photocatalyst particle 2, the photocatalyst activity of the photocatalyst composition 20 can be improved. Only one type of co-catalyst may be supported on the photocatalyst particle 2, or more than one type of co-catalyst may be supported. The co-catalyst 3 is preferably supported on the surface of the photocatalyst particle 2, but if the photocatalyst composition 20 is the photocatalyst dispersion liquid 10, the co-catalyst 3 may be dissolved in the dispersion medium 7.

Examples of metals contained in the co-catalyst 3 include platinum (Pt), gold (Au), silver (Ag), copper (Cu), zinc (Zn), palladium, iron, nickel, ruthenium, iridium, niobium, zirconium, and molybdenum. These metals may be included in the co-catalyst in the form of complexes, chlorides, bromides, iodides, oxides, hydroxides, sulfates, nitrates, carbonates, acetates, phosphates, or organic acids, for example. A suitable example of the co-catalyst is platinum. The photocatalyst particle 2 is preferably a tungsten oxide particle loaded with platinum as the co-catalyst 3.

A ratio (B/A) of weight B of the co-catalyst 3 to weight A of the photocatalyst particle 2 in the photocatalyst composition 20 can be (0.005/100) or more and (5/100) or less, and can be preferably (0.01/100) or more and (3/100) or less. If the ratio (B/A) is set to a lower limit value or less, the effect as a co-catalyst becomes small and high photocatalyst activity cannot be obtained. If the ratio (B/A) is set to an upper limit value or more, the amount of co-catalysts covering the surface of the photocatalyst particle increases, and the surface area of the photocatalyst particle in contact with the atmosphere becomes smaller. As a result, high photocatalyst activity cannot be obtained.

The water-soluble iodine compound 4 is an iodine compound that is readily soluble in water, such as metal iodide, ammonium iodide, iodic acid (HIO₃), and metal iodate. The metal iodide is, for example, potassium iodide, sodium iodide, and calcium iodide. The metal iodate is, for example, potassium iodate, sodium iodate, and potassium hydrogen iodate.

A ratio (D/C) of weight D of the iodine or water-soluble iodine compound to weight C of the photocatalyst particle 2 in the photocatalyst composition 20 can be (0.1/100) or more and (5/100) or less, and can be preferably (0.3/100) or more and (3/100) or less. If the ratio (D/C) is set to a lower limit value or less, the antibacterial properties of the photocatalyst composition 20 will decrease. If the ratio (D/C) is set to an upper limit value or more, the organic matter decomposition characteristics by the photocatalyst activity decrease.

FIG. 3 is an illustration of antibacterial action of a photocatalyst composite in the dark place and regeneration of iodine in the bright place.

Although details are not known, it is believed that when the photocatalyst composition 20 contains iodine (I₂), I₂ reacts with water (H₂O) to form highly reactive H₂OI⁺. When H₂OI⁺ comes in contact with a bacterium, it deactivates the bacterium by taking an electron away from (oxidizing) the bacterium. Thus, the photocatalyst composition 20 has antibacterial action both in the light and the dark. H₂OI⁺, which has taken an electron away from the bacterium, is reduced to form an iodide ion (I⁻). Therefore, the antibacterial action of the photocatalyst composition 20 in the dark is considered to gradually decrease.

When light hits (a bright place) the photocatalyst particle 2 contained in the photocatalyst composition 20 after H₂OI⁺ is oxidized and iodide ion (I⁻) is formed, photoexcitation generates an electron (e⁻) and a hole (h⁺) in the photocatalyst particle 2. The electron reacts with an adsorbate such as oxygen at the co-catalyst 3. The hole takes the electron away from I⁻ on the surface of the photocatalyst particle 2 and I⁻ is oxidized, resulting in I₂. This increases the amount of I₂ contained in the photocatalyst composition 20 in the light. This I₂ reacts with water to form H₂OI⁺, which exhibits antibacterial action. Thus, even when the photocatalyst composition 20 is placed for a long period of time in places where light and darkness are repeated (e.g., a place where brightness changes due to repetition of day and night), decrease in antibacterial action of the photocatalyst composition 20 during dark hours can be suppressed.

In addition, the photocatalyst composition 20 can have excellent antibacterial properties in the light due to both the action of hydroxyl radicals generated by oxidizing water through photocatalyst activity and the action of H₂OI⁺.

When the photocatalyst composition 20 contains metal iodide or ammonium iodide, the metal iodide or ammonium iodide is ionized on the surface of the photocatalyst particle 2, etc. to form an iodide ion (I⁻). This iodide ion is oxidized by a hole generated by the photocatalyst particle 2 receiving light, resulting in I₂. This I₂ becomes H₂OI⁺ in the light or dark and exhibits antibacterial action. H₂OI⁺, which has taken an electron away from a bacterium, is reduced to form an iodide ion (I⁻). The iodide ion (I⁻) becomes I₂ by photocatalyst activity. Thus, because iodine can be formed from an iodide ion by the photocatalyst activity, decrease in antibacterial action of the photocatalyst composition 20 during dark hours can be suppressed even when the photocatalyst composition 20 is placed for a long period of time.

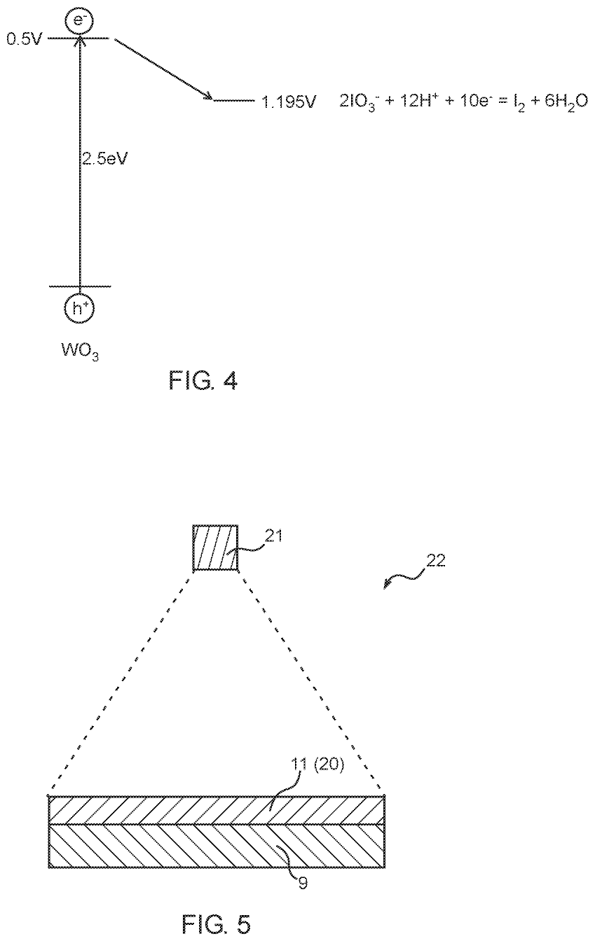

When the photocatalyst composition 20 contains iodic acid (HIO₃) or metal iodate, the iodic acid or metal iodate is ionized on the surface of the photocatalyst particle 2, etc. to form an iodate ion (IO₃⁻). This iodate ion reacts with an electron, a hydrogen ion (H+), etc. generated by the photocatalyst particle 2 receiving light to form iodine (I₂). FIG. 4 is an illustration of a reaction in which iodine is formed from an iodate ion by photocatalyst activity. A standard electrode potential for the reaction in which iodine is formed from an iodate ion is 1.195 V, and a conduction charge potential of the photocatalyst, tungsten oxide (WO3), is 0.5 V. That is, the standard electrode potential for the reaction in which iodine is formed from an iodate ion is about 0.695 V or more positively higher than the conduction charge potential of tungsten oxide. Thus, the reaction in which iodine is formed from an iodate ion by a photoexcited electron is promoted. In the dark and in the light, H₂OI⁺ generated from this I₂ acts and the photocatalyst composition 20 has antibacterial action. H₂OI⁺, which has taken an electron away from a bacterium, is reduced to form an iodide ion (I⁻). Also, in the light, a hole generated by the photocatalyst particle 2 receiving light takes the electron away from I⁻ on the surface of the photocatalyst particle 2, and I⁻ is oxidized to form I₂. Thus, since iodine can be generated from iodate and iodide ions by the photocatalyst activity, decrease in antibacterial action of the photocatalyst composition 20 during dark hours can be suppressed even when the photocatalyst composition 20 is placed for a long period of time.

When the photocatalyst composition 20 is the photocatalyst dispersion liquid 10, the photocatalyst dispersion liquid 10 contains the photocatalyst particle 2, the co-catalyst 3, the iodine or water-soluble iodine compound 4, and the dispersion medium 7.

The dispersion medium 7 is, for example, a polar solvent. Examples of the polar solvent include water, methanol, ethanol, and isopropanol. Also, the dispersion medium 7 may be an aqueous solvent. For example, the dispersion medium 7 can be a mixture of water and alcohol. At least one of the co-catalyst 3 and the iodine or water-soluble iodine compound 4 may be dissolved in the dispersion medium 7.

The percentage of the photocatalyst particle 2 in the photocatalyst dispersion liquid 10 is, for example, 0.1 wt % or more and 30 wt % or less, and is preferably 1.0 wt % or more and 20 wt % or less. If the percentage of the photocatalyst particle 2 is equal to or above an upper limit value, the viscosity of the photocatalyst dispersion liquid 10 increases and the thickness of the photocatalyst coating layer 11 formed by applying the photocatalyst dispersion liquid 10 will vary. This results in a large variation in the photocatalyst performance of the photocatalyst coating layer 11. If the percentage of the photocatalyst particle 2 is equal to or below a lower limit value, the antibacterial regeneration effect by photocatalyst activity (regeneration through the formation of I₂) becomes lower.

The pH of the photocatalyst dispersion liquid 10 is, for example, 1 or more and 7 or less, and is preferably 3.5 or more and 6.0 or less. If the pH is equal to or above an upper limit value, an iodine atom is less likely to exist in the form of I₂ and the antibacterial action is reduced. If the pH is equal to or below a lower limit value, the dispersibility of the photocatalyst particle 2 will decrease and the photocatalyst dispersion liquid 10 may corrode the substrate 9, limiting the scope of application.

The photocatalyst dispersion liquid 10 may contain a pH adjuster to adjust the pH within the range described above. Hydrochloric acid (HCl), sodium hydroxide (NaOH), etc. can be used as the pH adjuster. The photocatalyst dispersion liquid 10 may further contain a binder if necessary. In addition, the photocatalyst dispersion liquid 10 may further contain an additive if necessary.

When the photocatalyst composition 20 is the photocatalyst coating layer 11, the photocatalyst coating layer 11 can be formed by applying and drying the photocatalyst dispersion liquid 10 described above on the substrate 9. Coating methods are not limited, but include, for example, dip coating, spray coating, screen printing, spin coating, brush coating, roll coating, etc. The substrate 9 is, for example, glass, plastic, metal, ceramics, wood, stone, cement, concrete, fiber, filter, fabric, paper, leather, etc. When the co-catalyst 3, iodine or an iodine compound is dissolved in the dispersion medium 7, the co-catalyst 3, iodine or iodine compound is loaded on the surface of the photocatalyst particle 2 when a coating film of the photocatalyst dispersion liquid 10 dries.

When the photocatalyst composition 20 is the photocatalyst film 12, the photocatalyst film 12 can have, for example, a photocatalyst layer in which the photocatalyst composite 5 in which the co-catalyst 3 and iodine (I₂) or the water-soluble iodine compound 4 are supported on the surface of the photocatalyst particle 2 is dispersed in a resin 8 or a siloxane compound. The photocatalyst film 12 may have a single photocatalyst layer or a laminated structure with a photocatalyst layer on top of a base film.

The resin 8 is, for example, a UV-curing resin.

### Antibacterial Activation Apparatus

FIG. 5 is a schematic cross-sectional view of an antibacterial activation apparatus of the present embodiment.

An antibacterial activation apparatus 22 of the present embodiment includes a light source device 21 provided to irradiate light to the photocatalyst composition 20 including a photocatalyst particle, a co-catalyst, and a water-soluble iodine compound, and is characterized in that iodine is formed from an iodide ion by photocatalyst activity generated when the photocatalyst particle receives the light irradiated from the light source device 21.

The antibacterial activation apparatus 22 is an apparatus that activates the antibacterial properties of the photocatalyst composition 20. The antibacterial activation apparatus 22 may be an antibacterial regeneration apparatus that regenerates the reduced antibacterial properties of the photocatalyst composition 20.

The light source device 21 includes, for example, an LED element and a control unit with an LED driver that controls emission of light from the LED element. The light source device 21 is arranged to irradiate light to the photocatalyst composition 20.

As the light source device 21, the one that irradiates light with a wavelength of approximately 450 nm to the photocatalyst composition 20 can be used. The control unit with the LED driver is a drive unit that supplies drive power to the LED element to make it emit light, and is composed of an element that supplies voltage and current to the LED element.

The photocatalyst composition 20 that is illuminated by the light source device 21 may be a powder, a dispersion liquid, or a photocatalyst layer supported on a substrate. The antibacterial activation apparatus 22 can be installed, for example, to irradiate a photocatalyst filter with a photocatalyst layer with light from the light source device 21. The photocatalyst filter can be formed by using, for example, glass fibers processed to ensure air permeability to be made into a filter and loading photocatalysts on said filter. As a loading method, for example, the photocatalyst dispersion liquid 10 may be applied directly to the filter substrate, or the photocatalyst dispersion liquid 10 mixed with a binder, etc., which is a fixing material, may be applied to the filter substrate.

Specifically, when the photocatalyst filter is activated by light with a specific wavelength, an iodide ion is oxidized to iodine by photocatalyst activity in the light, thus regenerating the amount of iodine in the light, even if iodine is consumed by antibacterial action in the dark. This function allows the photocatalyst filter to be regenerated or activated.

An exposure time of light to the photocatalyst filter can be 12 to 48 hours. The wavelength of light to be applied to the photocatalyst filter can be 380 to 500 nm. An illuminance of light irradiation to the photocatalyst filter can be 500 to 24,000 lux.

### Iodine Solution

An iodine solution of the present embodiment contains iodine, which is an extract of the photocatalyst composition of the present embodiment after light irradiation. The iodine solution can be used, for example, as an antibacterial liquid, a disinfection liquid, a bactericide, an antifungal agent, and an iodine additive.

For example, if the photocatalyst composition 20 is a powder, an iodine solution can be obtained as a filtrate by filtering the photocatalyst composition 20 plus water.

### Preparation of Photocatalyst Slurry

A photocatalyst slurry was prepared by the following method. The photocatalyst slurry is used to prepare photocatalyst dispersion liquids of Examples 1 to 6 and Comparative Examples 1 to 3.

### (Primary Grinding Process)

Using a beads mill (Media-agitation, wet-type ultrafine grinding and dispersion machine “MSC50” manufactured by Nippon Coke & Engineering Co., Ltd.), 135 g of tungsten oxide (specifically, WO₃, manufactured by Kishida Chemical Co., Ltd.) and 1,215 g of ion exchanged water were mixed and primarily ground to obtain a dispersion liquid of tungsten oxide. The beads mill was equipped with beads (manufactured by Nikkato Corporation, diameter: 0.1 mm). The conditions of the beads mill were peripheral speed of 10 m/sec and processing time of 360 minutes. A primary particle diameter of the tungsten oxide contained in the dispersion liquid after the primary grinding was approximately 50 nm. The dispersion liquid after the primary grinding was used for a co-catalyst loading process without drying.

### (Co-Catalyst Loading Process)

Into the tungsten oxide dispersion liquid obtained in the primary grinding process, hydrogen hexachloroplatinate (IV) hexahydrate (manufactured by Kishida Chemical Co., Ltd., solid concentration: 98.5%) was dissolved. An amount of hydrogen hexachloroplatinate (IV) hexahydrate added was set such that the content of platinum alone is 0.025 wt % or 0.1 wt % relative to the mass of photocatalysts contained in a photocatalyst slurry to be prepared. The dispersion liquid was then heated at 100° C. to evaporate water. This resulted in a lump of platinum-loaded tungsten oxide. A lump of platinum-unloaded tungsten oxide was also prepared.

### (Secondary Grinding Process)

The lump obtained in the co-catalyst loading process was ground using a mortar and a pestle to obtain a ground material of tungsten oxide. The ground material was sieved using a vibrating sieve to obtain a tungsten oxide powder that has passed through a sieve with a 63 μm aperture. The resulting tungsten oxide powder was mixed with pure water to prepare a photocatalyst slurry containing 20 wt % tungsten oxide.

### Preparation of Photocatalyst Dispersion Liquid

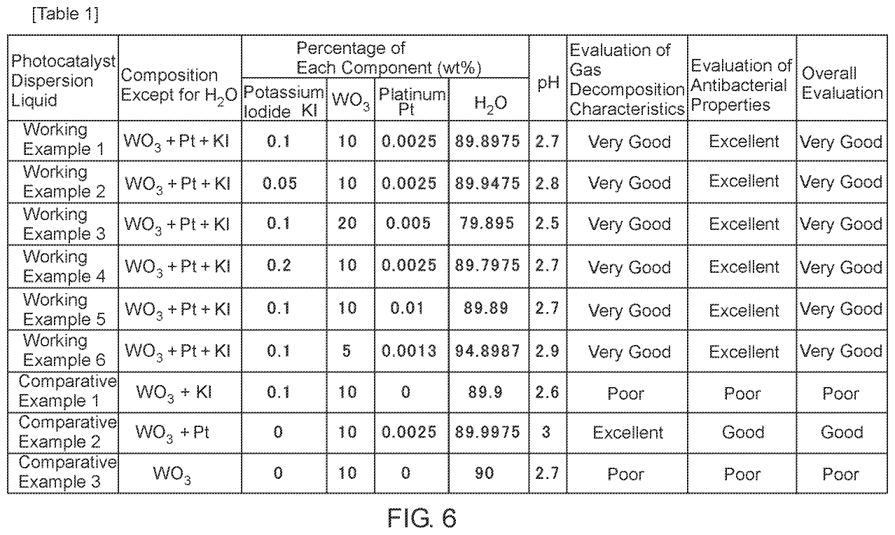

A dispersion liquid was prepared by adding water and potassium iodide to the prepared photocatalyst slurry and stirring, and then the dispersion liquid was irradiated with visible light of 450 nm wavelength and about 9,000 lux for 30 minutes while stirring to prepare photocatalyst dispersion liquids of Examples 1 to 6. Mixing ratios were adjusted to achieve component ratios shown in Table 1. A photocatalyst dispersion liquid of Comparative Example 1 was prepared by mixing a platinum-unloaded photocatalyst slurry with potassium iodide and water and irradiating it with visible light of 450 nm wavelength and about 9,000 lux for 30 minutes, a photocatalyst dispersion liquid of Comparative Example 2 was prepared by mixing a photocatalyst slurry with water and irradiating it with visible light of 450 nm wavelength and about 9,000 lux for 30 minutes, and a photocatalyst dispersion liquid of Comparative Example 3 was prepared by mixing a platinum-unloaded photocatalyst slurry with water and irradiating it with visible light of 450 nm wavelength and about 9,000 lux for 30 minutes. Mixing ratios were adjusted to achieve component ratios shown in Table 1 of FIG. 6.

### pH Measurement

A pH meter was used to measure pH of the photocatalyst dispersion liquids of Examples 1 to 6 and Comparative Examples 1 to 3. Measurement results are shown in Table 1 of FIG. 6.

### Acetaldehyde Gas Decomposition Experiment

Acetaldehyde gas decomposition experiments were conducted to evaluate the photocatalyst activity of each of the photocatalyst dispersion liquids of Examples 1 to 6 and Comparative Examples 1 to 3. Specifically, a photocatalyst dispersion liquid and a filter were placed in a petri dish (60 mm in diameter) in such quantities that the mass of photocatalyst was 0.1 g, and the photocatalyst dispersion liquid was dried at 80° C. for 30 minutes to form a photocatalyst filter in which a photocatalyst layer was supported on the filter. The petri dish containing the photocatalyst filter after drying was placed inside a 5 L capacity gas bag. The gas bag was filled with acetaldehyde gas at a concentration of 600 ppm. Light (central wavelength: 450 nm, illuminance: 4,500 lux) was then applied to the photocatalyst layer in the gas bag. An acetaldehyde concentration in the gas bag was measured before light irradiation (0.0 hour) and at 2, 4, 6, and 24 hours after the start of light irradiation. A gas detector tube for acetaldehyde (“92” manufactured by Gastec Corporation) was used to measure the acetaldehyde concentration. The higher the photocatalyst activity, the higher the acetaldehyde decomposition activity and the lower the acetaldehyde concentration.

An acetaldehyde decomposition rate was then calculated using the following equation.

Acetaldehyde decomposition rate (%)=(initial acetaldehyde concentration (600 ppm)−acetaldehyde concentration after light irradiation)/(initial acetaldehyde concentration (600 ppm))×100

Evaluations of acetaldehyde gas decomposition characteristics are shown in Table 1 of FIG. 6. In the gas decomposition characteristics in Table 1 of FIG. 6, an evaluation of a photocatalyst dispersion liquid (photocatalyst layer) in which the acetaldehyde decomposition rate was more than 80% 24 hours after the start of light irradiation is indicated by “Excellent”, an evaluation of a photocatalyst dispersion liquid (photocatalyst layer) in which the acetaldehyde decomposition rate was 40 to 80% 24 hours after the start of light irradiation is indicated by “Very Good”, and an evaluation of a photocatalyst dispersion liquid (photocatalyst layer) in which the acetaldehyde decomposition rate was less than 40% 24 hours after the start of light irradiation is indicated by “Poor”.

As can be seen from the evaluations of gas decomposition characteristics shown in Table 1, the photocatalyst layers prepared using the photocatalyst dispersion liquids of Examples 1-6 and Comparative Example 2 were found to have excellent acetaldehyde gas decomposition characteristics. In addition, the acetaldehyde gas decomposition characteristics of the photocatalyst layers prepared using the photocatalyst dispersion liquids of Comparative Examples 1 and 3 were found to be low.

This indicates that the photocatalyst layers containing platinum-loaded tungsten oxide have excellent acetaldehyde gas decomposition characteristics.

### Antibacterial Property Test 1

The antibacterial properties of each of the photocatalyst dispersion liquids of Examples 1 to 6 and Comparative Examples 1 to 3 were evaluated by conducting antibacterial property tests. Specifically, a centrifuge tube containing a photocatalyst dispersion liquid and a bacterial liquid (0.5 ml, E. coli) was set in a rotating incubator (BIOSPIN MBS-1 (manufactured by EYELA)) under room light, and the liquid in the centrifuge tube was agitated by rotating the rotating incubator (rotation speed: 60 rpm). At 30 minutes after the start of agitation, an additional bacterial liquid was added dropwise or a SCDLP medium was added to the centrifuge tube to be washed out by inverting and mixing. Then, 0.1 ml of the liquid in the centrifuge tube was applied to an agar medium, and the agar medium was placed in a 40° C. incubator for incubation. Based on the number of viable bacteria after incubation, an antibacterial activity value was calculated according to the following formula.

Antibacterial activity value=log (initial viable bacteria count after incubation per sample)−log (viable bacteria count after reaction per sample)

Based on the calculated antibacterial activity values, the antibacterial properties of the photocatalyst dispersion liquids of Examples 1 to 6 and Comparative Examples 1 to 3 were evaluated.

Evaluation results are shown in Table 1 of FIG. 6. An evaluation of a photocatalyst dispersion liquid with an antibacterial activity value of 4 or higher is indicated by “Excellent”, an evaluation of a photocatalyst dispersion liquid with an antibacterial activity value of 1 to 2 is indicated by “Good”, and a n evaluation of a photocatalyst dispersion liquid with an antibacterial activity value of lower than 1 is indicated by “Poor”. An evaluation of a photocatalyst dispersion liquid with an antibacterial activity value of 2 to 3 was indicated by “Very Good”, but there was no applicable photocatalyst dispersion liquid.

The photocatalyst dispersion liquids of Examples 1 to 6 were found to have excellent antibacterial properties. On the other hand, the photocatalyst dispersion liquids in Comparative Examples 1 to 3 were evaluated as “Good” or “Poor” for antibacterial properties. Therefore, it was found that the photocatalyst dispersion liquid has excellent antibacterial properties when it contains tungsten oxide, platinum and potassium iodide.

Table 1 of FIG. 6 also shows an overall evaluation of each photocatalyst dispersion liquids. The overall evaluation was aligned with the lower of the gas decomposition characteristic evaluation and the antibacterial property evaluation.

### Preparation of Photocatalyst Filter

Photocatalyst filters of Example 7 and Comparative Example 4 were prepared. Specifically, a photocatalyst dispersion liquid and a filter were placed in a petri dish (60 mm in diameter) in such quantities that the mass of photocatalyst was 0.1 g, and the photocatalyst dispersion liquid was dried at 80° C. for 30 minutes to form a photocatalyst filter in which a photocatalyst layer was supported on the filter. The photocatalyst dispersion liquid of Example 1 was used to make the photocatalyst filter of Example 7, and the photocatalyst dispersion liquid of Comparative Example 2 was used to make the photocatalyst filter of Comparative Example 4.

### Antibacterial Property Test 2

The prepared photocatalyst filters of Example 7 and Comparative Example 4 were used to conduct an antibacterial property test.

Specifically, a centrifuge tube containing 1 g of photocatalyst filter and a bacterial liquid (0.5 ml, E. coli) was set in a rotating incubator (BIOSPIN MBS-1 (manufactured by EYELA)) under room light, and the liquid in the centrifuge tube was agitated by rotating the rotating incubator (rotation speed: 60 rpm). At 30 minutes after the start of agitation, an additional bacterial liquid was added dropwise or a SCDLP medium was added to the centrifuge tube to be washed out by inverting and mixing. Then, 0.1 ml of the liquid in the centrifuge tube was applied to an agar medium, and the agar medium was placed in a 40° C. incubator for incubation. Based on the number of viable bacteria after incubation, an antibacterial activity value was calculated according to the following formula.

Antibacterial activity value=log (initial viable bacteria count after incubation per sample)−log (viable bacteria count after reaction per sample)

Based on the calculated antibacterial activity values, the antibacterial properties of the photocatalyst filters of Example 7 and Comparative Example 4 were evaluated.

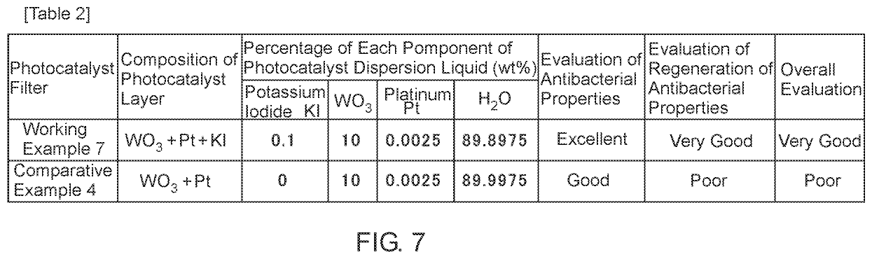

Evaluation results are shown in Table 2 of FIG. 7. An evaluation of a photocatalyst filter with an antibacterial activity value of 4 or higher is indicated by “Excellent”, while an evaluation of a photocatalyst filter with an antibacterial activity value of 1 to 2 is indicated by “Good”. A photocatalyst filter with an antibacterial activity value of lower than 1 was evaluated as “Poor”, and a photocatalyst filter with an antibacterial activity value of 2 to 3 was evaluated as “Very Good”, but there was no applicable photocatalyst filter.

### Antibacterial Regeneration Experiment

The antibacterial properties of each of the photocatalyst filters of Example 7 and Comparative Example 4 were evaluated by conducting an antibacterial regeneration test. Specifically, after the photocatalyst filter of Example 7 or Comparative Example 4 was introduced into a regeneration device after three repeated antibacterial property tests, the photocatalyst filter was irradiated with visible light of approximately 9,000 lux for 24 hours by a 450 nm wavelength LED. Then, the photocatalyst filter after light irradiation was tested for antibacterial properties. Based on test results of this antibacterial property test, antibacterial activity values were calculated according to the following formula.

Antibacterial activity value=log (initial viable bacteria count after incubation per sample)−log (viable bacteria count after reaction per sample)

Evaluation results are shown in Table 2 of FIG. 7. In Table 2, an evaluation of a photocatalyst filter with an antibacterial activity value of 2 to 3 is indicated by “Very Good”, while an evaluation of a photocatalyst filter with an antibacterial activity value of lower than 1 is indicated by “Poor”. An evaluation of a photocatalyst filter with an antibacterial activity value of 4 or higher was indicated by “Excellent”, and an evaluation of a photocatalyst filter with an antibacterial activity value of 1 to 2 was indicated by “Good”, but there was no applicable photocatalyst filter.

These results show that the photocatalyst filter of Example 7 has excellent antibacterial properties. It was also found that by treating the photocatalyst filter of Example 7 in the regeneration device after repeating the antibacterial property test three times, the photocatalyst filter of Example 7 had antibacterial properties again. On the other hand, the antibacterial properties of the photocatalyst filter of Comparative Example 4 were evaluated as “Good”, and the antibacterial properties of the photocatalyst filter of Comparative Example 4 after repeating the antibacterial property test three times and then after being treated in the regeneration device was evaluated as “Poor”. Therefore, it was found that the photocatalyst filter has excellent antibacterial regenerative properties when the photocatalyst filter contains tungsten oxide, platinum, and potassium iodide.

### Reference Signs List

2: photocatalyst particle, 3: co-catalyst, 4: iodine or water-soluble iodine compound, 5: photocatalyst composite, 7: dispersion medium, 8: resin, 9: substrate, 10: photocatalyst dispersion liquid, 11: photocatalyst coating layer, 12: photocatalyst film, 13: container, 20: photocatalyst composition, 21: light source device, 22: antibacterial activation apparatus

What is claimed is:

1. A photocatalyst composition comprising a photocatalyst particle, a co-catalyst, and iodine or a water-soluble iodine compound, wherein the iodine is formed from an iodide ion by photocatalyst activity generated by the photocatalyst particle receiving light.

2. The photocatalyst composition according to claim 1, wherein the water-soluble iodine compound is metal iodide, ammonium iodide, iodic acid or metal iodate.

3. The photocatalyst composition according to claim 1, wherein the co-catalyst is supported on a surface of the photocatalyst particle.

4. The photocatalyst composition according to claim 1, wherein the iodine or water-soluble iodine compound is supported on a surface of the photocatalyst particle.

5. The photocatalyst composition according to claim 1, wherein the photocatalyst particle comprises tungsten oxide.

6. The photocatalyst composition according to claim 1, wherein the co-catalyst is a metal or metal oxide containing at least platinum.

7. The photocatalyst composition according to claim 1, wherein a ratio (B/A) of weight B of the co-catalyst to weight A of the photocatalyst particle in the photocatalyst composition is (0.005/100) or more and (5/100) or less.

8. The photocatalyst composition according to claim 1, wherein a ratio (D/C) of weight D of the iodine or water-soluble iodine compound to weight C of the photocatalyst particle in the photocatalyst composition is (0.1/100) or more and (5/100) or less.

9. An iodine solution comprising iodine, which is an extract of the photocatalyst composition according to claim 1 after light irradiation.

10. A photocatalyst dispersion liquid comprising a photocatalyst particle, a co-catalyst, iodine or a water-soluble iodine compound, and a dispersion medium.

11. The photocatalyst dispersion liquid according to claim 10, wherein a percentage of the photocatalyst particle in the photocatalyst dispersion liquid is 0.1 wt % or more and 30 wt % or less.

12. The photocatalyst dispersion liquid according to claim 10, further comprising a pH adjuster.

13. The photocatalyst dispersion liquid according to claim 10, wherein pH of the photocatalyst dispersion liquid is 1 or more and 7 or less.

14. An antibacterial activation method comprising irradiating a photocatalyst composition containing a photocatalyst particle, a co-catalyst, and a water-soluble iodine compound with light,
wherein iodine is formed from an iodide ion by photocatalyst activity generated by the photocatalyst particle receiving light.

15. The antibacterial activation method according to claim 14, further comprising extracting iodine from the photocatalyst composition after light irradiation.

16. An antibacterial activation apparatus comprising a light source device configured to irradiate a photocatalyst composition containing a photocatalyst particle, a co-catalyst, and a water-soluble iodine compound with light, wherein iodine is formed from an iodide ion by photocatalyst activity generated by the photocatalyst particle receiving light irradiated from the light source device.
